(19) United States
(12) Patent Application Publication
(10) Pub. No.: US 2026/0215980 A1
(43) Pub. Date: Jul. 30, 2026

(54) ROBOTIC SYSTEM FOR OPHTHALMIC INJECTION FACILITY

(71) Applicant: Alcon Inc., Fribourg (CH)

(72) Inventors: Patrick Terry, Goleta, CA (US); Paul R. Hallen, Colleyville, TX (US)

(21) Appl. No.: 19/456,606

(22) Filed: Jan. 22, 2026

Related U.S. Application Data
(60) Provisional application No. 63/750,896, filed on Jan. 29, 2025.

(51) Int. Cl. A61G 5/04; A61F 9/00; A61B 34/30

(52) U.S. Cl. CPC A61G 5/04; A61F 9/0008; A61B 34/30; A61G 2203/22; A61G 2203/30; A61G 2210/00

(57) ABSTRACT

In certain embodiments, a robotic system for an ophthalmic injection facility includes one or more patient transportation robots and an ophthalmic injection robot located in a treatment hub of the ophthalmic injection facility. Each patient transportation robot includes a drive system, one or more sensors, a patient interface (PI) system, a chair, and a control system configured to navigate to the treatment hub based on the sensor data. The ophthalmic injection robot includes a support frame, a robotic arm, and a control system. The robotic arm includes one or more cameras and a docking assembly including a needle. The control system is configured to position the docking assembly to couple the docking assembly to a PI device attached to an eye of the patient, inject a drug through the needle into the eye of the patient, and position the docking assembly to decouple the docking assembly from the PI device.

### CROSS-REFERENCE TO RELATED APPLICATIONS

This application claims the benefit of U.S. Provisional Application Serial No. 63/750,896 (filed on January 29, 2025), the content of which is incorporated by reference herein in its entirety.

### BACKGROUND

The present disclosure relates to a robotic system, and, more particularly, to a robotic system for an ophthalmic injection facility.

An ophthalmic injection delivers medication directly into the eye of a patient using a needle. The structures of the eye are extremely small and delicate and, therefore, ophthalmic injections are performed by skilled ophthalmic surgeons. Becoming an ophthalmic surgeon requires many years of training, and the time spent by the ophthalmic surgeon in the treatment room of an ophthalmic injection facility is therefore a very valuable resource.

### SUMMARY

In certain embodiments, a robotic system for an ophthalmic injection facility comprises one or more patient transportation robots, and an ophthalmic injection robot located in a treatment hub of the ophthalmic injection facility.

In certain embodiments, each patient transportation robot comprises a drive system, one or more sensors configured to generate sensor data, a patient interface (PI) system configured to be coupled to a PI device, a chair configured to support a patient during an ophthalmic injection procedure, and a control system configured to navigate to the treatment hub based on the sensor data.

In certain embodiments, the ophthalmic injection robot comprises a support frame configured to receive the patient transportation robot, a robotic arm coupled to the support frame, and a control system. The robotic arm comprises one or more cameras and a docking assembly including a needle. The control system is configured to position the docking assembly to couple the docking assembly to a PI device attached to an eye of the patient, inject a drug through the needle into the eye of the patient, and position the docking assembly to decouple the docking assembly from the PI device attached to the eye of the patient.

### BRIEF DESCRIPTION OF THE DRAWINGS

FIG. 1A depicts a schematic diagram of an ophthalmic injection facility, in accordance with embodiments of the present disclosure.

FIG. 1B depicts a block diagram for a control computer for the ophthalmic injection facility, in accordance with embodiments of the present disclosure.

FIG. 2A depicts a patient transportation robot for the ophthalmic injection facility, in accordance with embodiments of the present disclosure.

FIG. 2B depicts a block diagram of the patient transportation robot depicted in FIG. 2A, in accordance with embodiments of the present disclosure.

FIG. 2C depicts a user interface (UI) for the patient transportation robot depicted in FIG. 2A, in accordance with embodiments of the present disclosure.

FIG. 3A depicts an ophthalmic injection robot for the ophthalmic injection facility, in accordance with embodiments of the present disclosure.

FIG. 3B depicts a top view of a docking assembly of the ophthalmic injection robot, in accordance with embodiments of the present disclosure

FIG. 3C depicts a partial side view of the docking assembly depicted in FIG. 3B, in accordance with embodiments of the present disclosure

FIG. 3D depicts a block diagram of the ophthalmic injection robot depicted in FIG. 3A, in accordance with embodiments of the present disclosure.

FIGS. 4A, 4B depict a PI device for an ophthalmic injection robot, in accordance with embodiments of the present disclosure.

### DETAILED DESCRIPTION

Ophthalmic injections (such as intravitreal injections, etc.) may be used to treat certain conditions, such as age-related macular degeneration (AMD), diabetic eye disease, retinal vein occlusion, etc. While the number of patients that receive monthly ophthalmic injections has been increasing, there are not enough skilled ophthalmic surgeons and ophthalmic injection facilities to keep up with this growing demand. Additionally, the number of drugs used for ophthalmic injections may increase by a factor of 4 (or more) over the next 10 years. Unfortunately, the number of patients, the number of drugs, and the limited number of skilled ophthalmic surgeons and ophthalmic injection facilities produce inefficiencies, complexities, and mistakes when preparing the patient and delivering the ophthalmic injection in an ophthalmic injection facility.

Embodiments of the present disclosure advantageously provide a robotic system for an ophthalmic injection facility that improves patient throughput, reduces complexity, and reduces or minimizes mistakes.

The robotic system may include patient transportation robots and an ophthalmic injection robot. Each patient transportation robot conveys a patient through a sequence of procedure hubs within the treatment room of the ophthalmic injection facility, such as a diagnostic hub, an anesthetic hub, a patient interface hub, an antiseptic hub, and a treatment hub. The ophthalmic injection robot is located in the treatment hub, and administers the ophthalmic injections, under the supervision and control of an ophthalmic surgeon, to each patient on each patient transportation robot. The ophthalmic injections may include injections into the anterior segment or the posterior segment of the eye (such as intravitreal injections, subretinal injections, suprachoroidal injections, etc.), as well as injections around the eye (such as retrobulbar, peribulbar, etc.).

In certain embodiments, patient information (such as the patient’s name, history, etc.), diagnostic measurements (such as optical coherence tomography (OCT) measurements, intraocular pressure (IOP) measurements, etc.), biometric data, the patient’s treatment plan, the ophthalmic surgeon’s name, etc., may be stored electronically and presented on a touchscreen display of each patient transportation robot. Additionally, the type and quantity of the drugs that are administered to the patient (such as an antibiotic, an anesthetic, the injected drug, etc.) may be tracked electronically to avoid complications, mistakes, etc., as well as to manage the consumable inventory of the ophthalmic injection facility. For example, a UI may be presented on the touchscreen display that includes controls and data that are associated with the administration of the anesthesia to the patient prior to the ophthalmic injection, such as a start widget, an elapsed time display bar, a patient ready icon, etc.

In some embodiments, the robotic system may include one patient transportation robot and an ophthalmic injection robot.

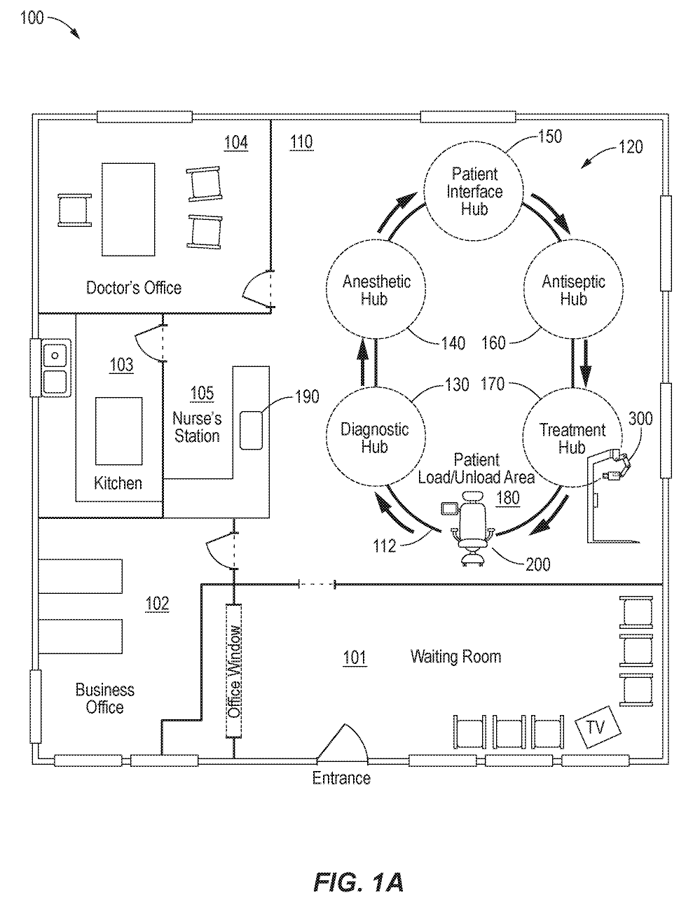

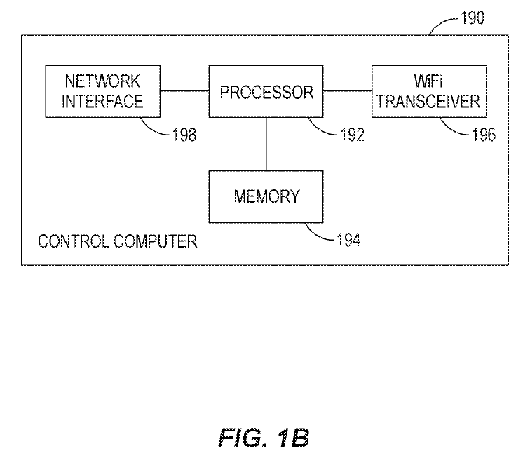

FIG. 1A depicts a schematic diagram of an ophthalmic injection facility 100, in accordance with embodiments of the present disclosure.

In certain embodiments, the ophthalmic injection facility 100 may include a waiting room 101, a business office 102, a kitchen 103, a doctor’s office 104, a nurse’s station 105, and a treatment room 110. The treatment room 110 includes patient transportation robots 200 that convey patients between procedure hubs 120. In certain embodiments, a facility staff member (such as a nurse, a nursing assistant, a medical technician, etc.) or the ophthalmic surgeon is stationed at each procedure hub 120 in order to perform a specific activity of the ophthalmic injection procedure on each patient as the patient transportation robots 200 convey the patients through the procedure hubs 120. Each patient remains seated on a patient transportation robot 200 during the entire ophthalmic injection procedure. In some embodiments, a facility staff member may be assigned to two procedure hubs 120, depending on the timing of the movement of the patient transportation robots 200 through the procedure hubs 120.

In certain embodiments, the procedure hubs 120 include a diagnostic hub 130, an anesthetic hub 140, a patient interface hub 150, an antiseptic hub 160, and a treatment hub 170. A patient loading/unloading area 180 is generally located between the diagnostic hub 130 and the treatment hub 170. A patient enters the treatment room 110 from the waiting room 101, mounts a patient transportation robot 200 that is located in the patient loading/unloading area 180, and is conveyed to the diagnostic hub 130 to begin the diagnostic activity of the ophthalmic injection procedure. The patient is then conveyed to the anesthetic hub 140 (for administration of an anesthetic), the patient interface hub 150 (for installation of a PI device), the antiseptic hub 160 (for administration of an antiseptic), and the treatment hub 170 (for administration of the ophthalmic injection).

After the ophthalmic injection has been administered by the ophthalmic injection robot 300 in the treatment hub 170, the patient is conveyed to the patient loading/unloading area 180, dismounts from the patient transportation robot 200, and exits the treatment room 110 to the waiting room 101. While the procedure hubs 120 are depicted as being arranged in an oval shape, other arrangements are also supported, such as a circular shape, a straight line, a star shape, etc.

The diagnostic hub 130 may include, inter alia, an imaging system, such as an OCT system, that is operated by a medical technician to provide data associated with the patient’s eye to be treated, such as image data, measurement data, etc. The data may be transmitted to the control

computer 190, which may store the data in memory for later access during the activities at other procedure hubs 120.

The anesthetic hub 140 may include a supply of topical anesthetic that may be administered by a nurse directly over the cornea and conjunctiva of each patient using drops or gels, such as lidocaine, proparacaine, tetracaine, etc. The same dosage of topical anesthetic may be used for each patient, so the consumption may be monitored by the control computer 190 as each patient transportation robot 200 leaves the anesthetic hub 140.

The patient interface hub 150 may include a supply of PI devices that may be attached by a nurse directly to the cornea of each patient. One PI device may be used for each patient, so the consumption may be monitored by the control computer 190 as each patient transportation robot 200 leaves the patient interface hub 150.

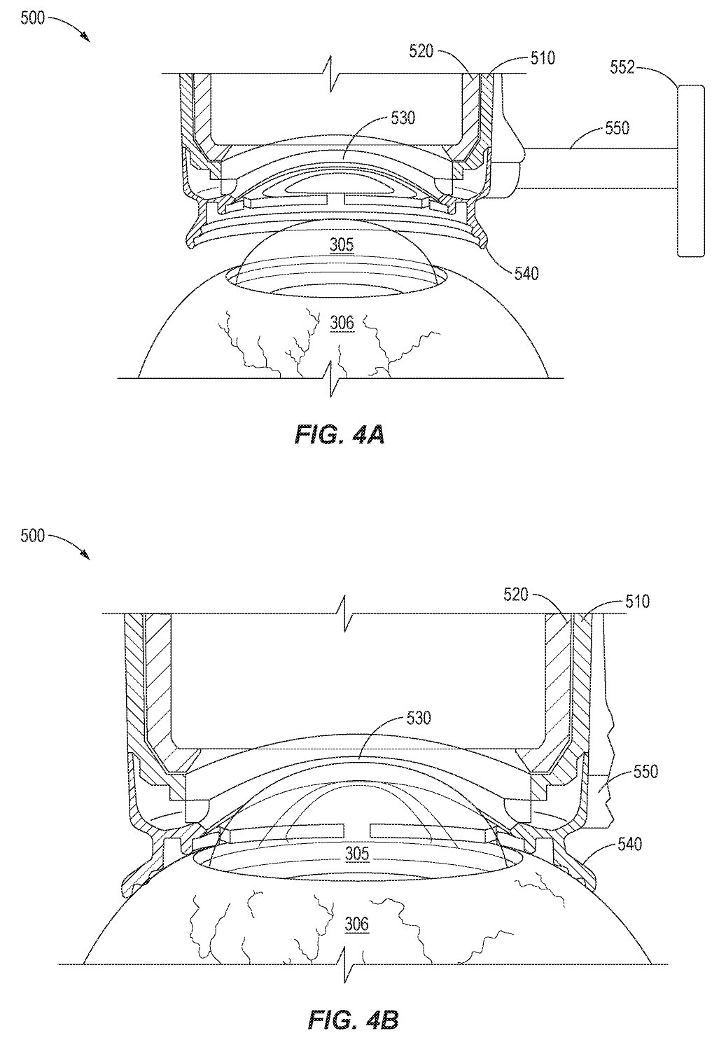

Referring to FIG. 4A, a PI device 500 is depicted before attachment to the cornea 305 of the eye 306 of the patient. In certain embodiments, the PI device 500 may include an outer body 510, an inner body 520, an applanation lens 530, a flexible suction ring 540, and a suction tube 550. The suction tube 550 is coupled to the suction ring 540, and includes a device connector 552. The device connector is configured to attach to a PI system connector on each patient transportation robot 200. The PI system connector is coupled to a vacuum pump on the patient transportation robot 200, which provides suction to the suction ring 540 when the device connector 552 is attached to the PI system connector.

Referring to FIG. 4B, the PI device 500 has been attached to the cornea 305 of the eye 306 of the patient. The vacuum pump on the patient transportation robot 200 is providing suction (such as a partial vacuum) to the suction ring 540, which applanates the cornea 305. The PI device 500 prevents the eye 306 from moving during the ophthalmic injection by the ophthalmic injection robot 300 in the treatment hub 170.

The antiseptic hub 160 may include a supply of topical antiseptic that may be administered by a nurse directly to the eye of each patient using drops, such as povidone-iodine (PI), etc. The same dosage of topical antiseptic may be used for each patient, so the consumption may be monitored by the control computer 190 as each patient transportation robot 200 leaves the antiseptic hub 160.

In some embodiments, the locations of the anesthetic hub 140 and the patient interface hub 150 may be switched, so that the topical antiseptic may be administered prior to the attachment of the PI device 500. In other embodiments, the activities in two procedure hubs 120 may be combined into a single procedure hub 120, such as the patient interface hub 150 and the antiseptic hub 160. For example, the topical antiseptic that may be administered, and then the PI device 500 may be attached in the same procedure hub.

The treatment hub 170 may include, inter alia, the ophthalmic injection robot 300 to inject the ophthalmic drug into the eye of the patient. After the patient transportation robot 200 arrives in the treatment hub 170, the patient transportation robot 200 docks to the ophthalmic injection robot 300, the patient’s head is clamped in place by the ophthalmic injection robot 300, and the PI device 500 is then docked to the ophthalmic injection robot 300. The ophthalmic injection robot 300 then injects the ophthalmic drug into the eye of the patient under the supervision and control of the ophthalmic surgeon. The ophthalmic injection robot 300 is then undocked from the PI device 500, and the patient transportation robot 200 is undocked from the ophthalmic injection robot 300 and travels to the patient loading/unloading area 180.

In certain embodiments, the treatment room 110 may include one patient transportation robot 200 for each procedure hub 120, so that a different activity may be being simultaneously performed on each patient at the same time. For example, the treatment room 110 depicted in FIG. 1A includes five (5) procedure hubs 120, so five (5) patient transportation robots 200 may be provided. In another example, at least three (3) patient transportation robots 200 may be provided for five (5) procedure hubs 120, etc. In some embodiments, the time to perform the activities in each procedure hub 120 may be different, so a fewer number of patient transportation robots 200 may be needed, such as four (4) patient transportation robots 200 for five (5) procedure hubs 120, etc.

Additionally, the anesthetic administered to the patient in the anesthetic hub 140 may require a certain period of time to become effective, such as 10 minutes, etc. Accordingly, the movement of the patient transportation robots 200 may be coordinated so that the time between the administering of the anesthetic to a patient in the anesthetic hub 140 and the arrival of the patient in the treatment hub 170 (via the patient interface hub 150 and the antiseptic hub 160) is not less than the effective time of the anesthetic.

The control computer 190 may be located at the nurse’s station 105. The control computer 190 may be configured to create wireless communication links to communicate with the patient transportation robots 200, such as WiFi communication links, Bluetooth communication links, etc. Generally, the processor of the control computer 190 is configured to control the movement of each patient transportation robot 200 between the diagnostic hub 130, the anesthetic hub 140, the patient interface hub 150, the antiseptic hub 160, and the treatment hub 170. The control computer 190 may also be configured to communicate with the ophthalmic injection robot 300 over a wireless network (such as WiFi, Bluetooth, etc.) or a wired network (such as Ethernet, etc.).

In certain embodiments, the control computer 190 may transmit a “travel” command to the a particular patient transportation robot 200 over a wireless communication link. The travel command may direct the patient transportation robot 200 to move to the next procedure hub 120, may include an identifier of the next procedure hub 120 (such as the diagnostic hub 130, etc.). After arriving at the next procedure hub 120, the patient transportation robot 200 may transmit a travel response to the control computer 190 to confirm arrival at the next procedure hub 120. Similarly, the travel command may direct the patient transportation robot 200 to move to the patient loading/unloading area 180, and, after arriving at the patient loading/unloading area 180, the patient transportation robot 200 may transmit a travel response to the control computer 190 to confirm arrival at the patient loading/unloading area 180.

In some embodiments, the identifier may be presented on a touchscreen display of the patient transportation robot 200 for presentation to the facility staff or ophthalmic surgeon. The facility staff member or surgeon may select the identifier to confirm that the patient conveyed by the patient transportation robot 200 has arrived in the correct procedure hub 120, which may be transmitted to the control computer 190.

In certain embodiments, the control computer 190 may transmit a “pause” command to one (or more) of the patient transportation robots 200 over a wireless communication link. The pause command may direct the patient transportation robot 200 to stop moving. After stopping, the patient transportation robot 200 may transmit a pause response to the control computer 190 to confirm that the patient transportation robot 200 has stopped. Similarly, the control computer 190 may transmit a “resume” command to the patient transportation robot 200 over a wireless communication link. The resume command may direct the patient transportation robot 200 to resume moving to the next location, such as the next procedure hub 120 or the patient loading/unloading area 180. After the patient transportation robot 200 begins moving, the patient transportation robot 200 may transmit a resume response to the control computer 190 to confirm that the patient transportation robot 200 has resumed moving. After arriving at the next procedure hub 120 (or the patient loading/unloading area 180), the patient transportation robot 200 may transmit a travel response to the control computer 190 to confirm arrival.

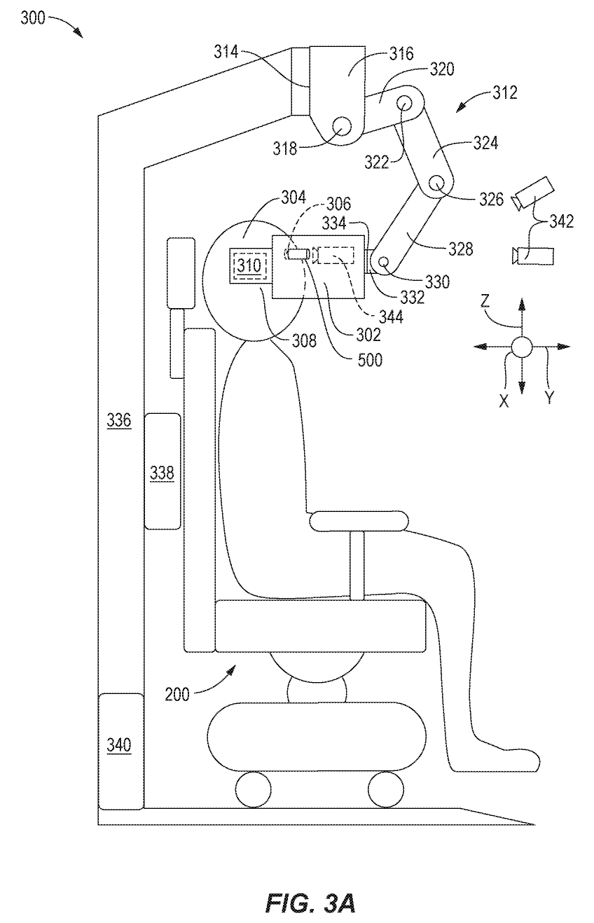

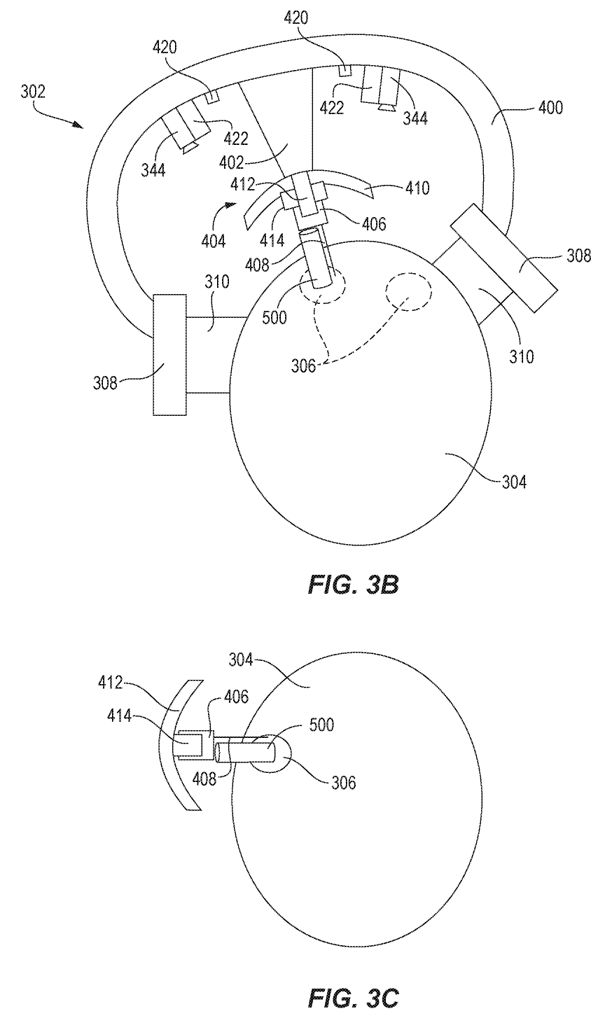

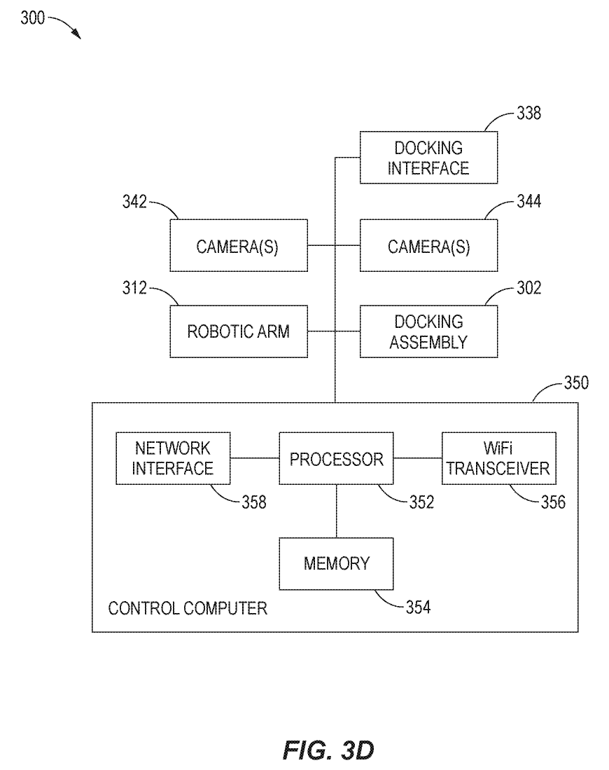

In certain embodiments, the control computer 190 may transmit a “dock” command to a patient transportation robot 200 located in the treatment hub 170 over a wireless communication link. The dock command may direct the patient transportation robot 200 to dock with a docking interface 338 (see FIG. 3A) on the ophthalmic injection robot 300. After docking with the ophthalmic injection robot 300, the patient transportation robot 200 may transmit a dock response to the control computer 190 to confirm that the patient transportation robot 200 has docked with the ophthalmic injection robot 300.

In some embodiments, a travel command that identifies the treatment hub 170 as the next procedure hub 120 may be followed immediately by a dock command, so that the patient transportation robot 200 moves to the treatment hub 170 and immediately docks with the ophthalmic injection robot 300.

In certain embodiments, the control computer 190 may transmit an “undock” command to a patient transportation robot 200 located in the treatment hub 170 over a wireless communication link. The undock command may direct the patient transportation robot 200 to undock from the ophthalmic injection robot 300. After undocking from the ophthalmic injection robot 300, the patient transportation robot 200 may transmit an undock response to the control computer 190 to confirm that the patient transportation robot 200 has undocked with the ophthalmic injection robot 300.

In some embodiments, an undock command may be followed immediately by a travel command that identifies the patient loading/unloading area 180 as the next procedure hub 120, so that the patient transportation robot 200 undocks from the ophthalmic injection robot 300 and immediately moves to the patient loading/unloading area 180.

In certain embodiments, the control computer 190 may be configured to prevent more than one patient transportation robot 200 from being present in the diagnostic hub 130, the anesthetic hub 140, the patient interface hub 150, the antiseptic hub 160, the treatment hub 170, or the patient loading/unloading area 180 at the same time. Additionally, the control computer 190 may be configured to control a time of movement of each patient transportation robot 200 between the anesthetic hub 140 and the treatment hub 170 (via the patient interface hub 150 the antiseptic hub 160). For example, the time of movement may be the length of time required for the anesthetic to take effect on a typical patient, such as 6 minutes, 8 minutes, at least 10 minutes, etc.

Advantageously, the control computer 190 may track the type and quantity of the drugs that are administered to the patient (such as an antibiotic, an anesthetic, the injected drug, etc.) to avoid complications, mistakes, etc., as well as to manage the consumable inventory of the ophthalmic injection facility.

In certain embodiments, a continuous navigation line 112 may be provided on the floor of the ophthalmic injection facility 100 to delineate the path or track that link the procedure hubs 120 and the patient loading/unloading area 180. In some embodiments, a continuous rail may replace the navigation line 112 on the floor of the ophthalmic injection facility 100, and each patient transportation robot 200 may translate along the rail to navigate to the procedure hubs 120 and the patient loading/unloading area 180.

FIG. 1B depicts a block diagram for a control computer 190 for the ophthalmic injection facility 100, in accordance with embodiments of the present disclosure.

In certain embodiments, the control computer 190 may include a processor 192 coupled to a memory 194, a wireless transceiver such as a WiFi transceiver 196, and a network interface 198. The WiFi transceiver 196 is coupled to a WiFi antenna, and the network interface 198 may be coupled to a wired network (such as Ethernet, etc.).

The processor 192 may be configured to generate and transmit various commands, such as the travel command, the pause command, the dock command, the undock command, etc., and to receive various responses, such as the travel response, the pause response, the dock response, the undock response, etc. The processor 192 may also be configured to track the consumables used at each procedure hub 120 through the travel responses, the undock responses, etc.

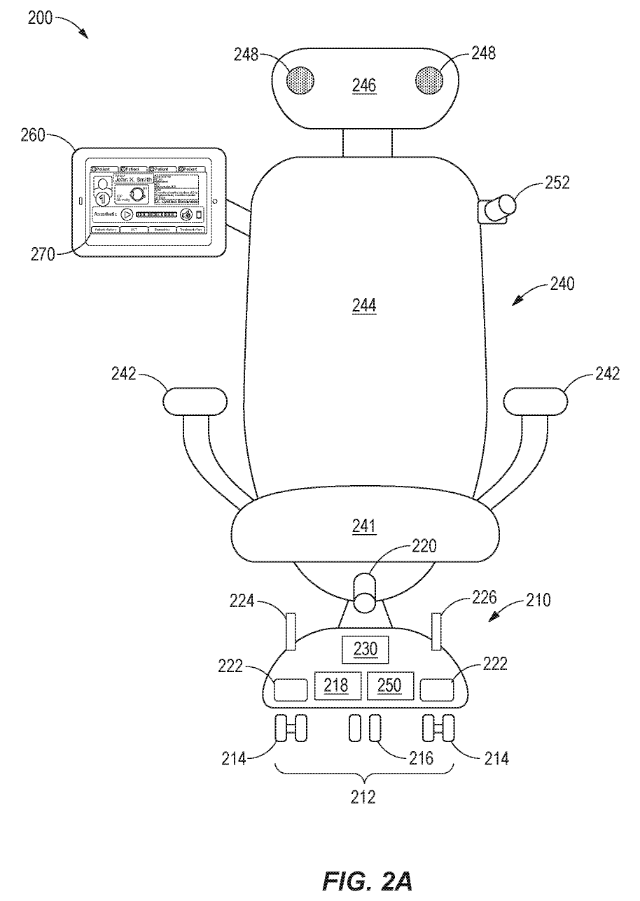

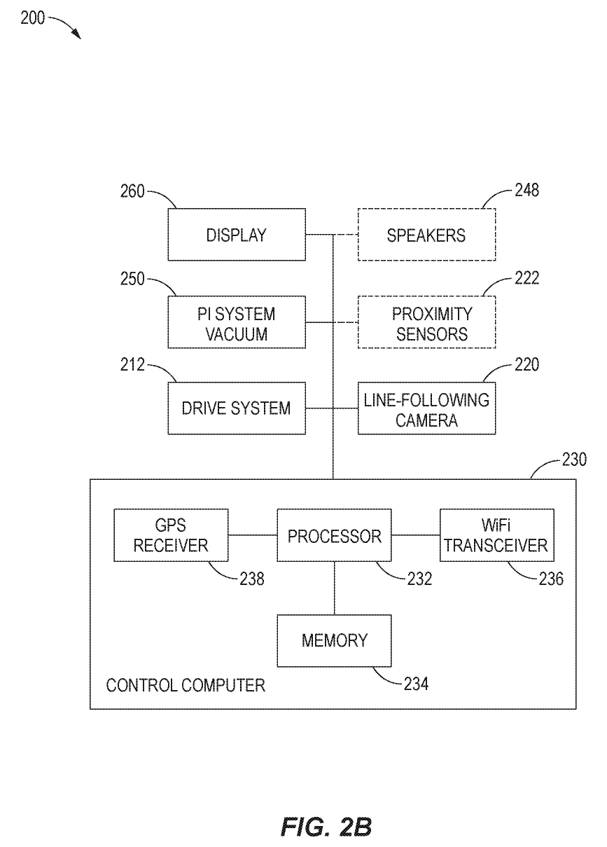

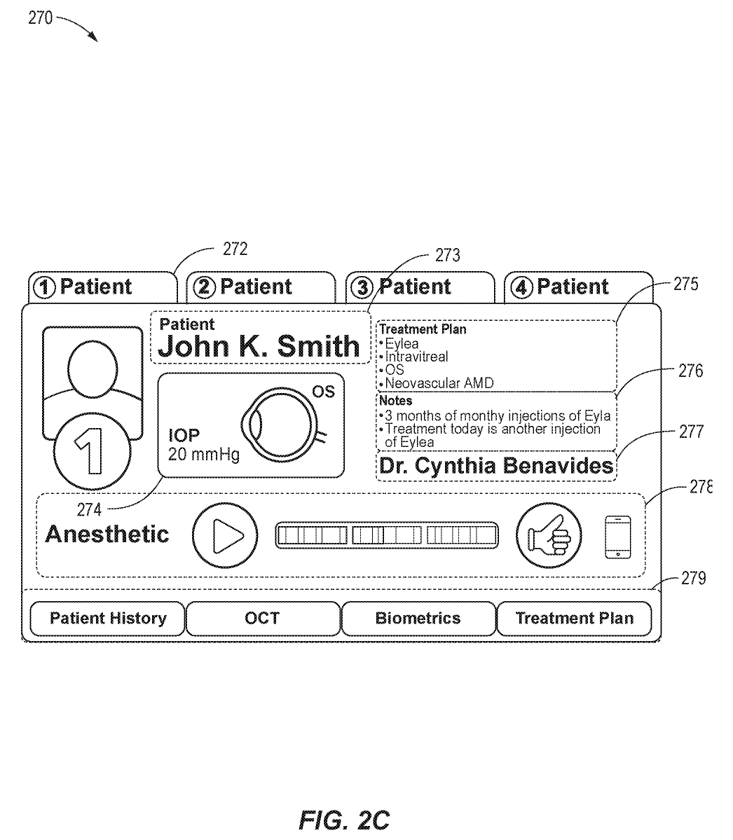

FIG. 2A depicts a patient transportation robot 200, in accordance with embodiments of the present disclosure.

In certain embodiments, the patient transportation robot 200 may include, inter alia, a chassis 210, a drive system 212, a battery 218, a line-following camera 220, a WiFi antenna 224, a global positioning system (GPS) antenna 226, a control system 230, a chair assembly 240, a PI system, and a touchscreen display 260 for presenting UI 270. The chair assembly 240 may include a seat 241, armrests 242, a back 244, and a headrest 246. The chair assembly 240 may also include a harness to secure the patient to the seat 241. The PI system may include a vacuum pump 250 and a system connector 252. In some embodiments, the patient transportation robot 200 may include one or more proximity sensors 222 in the chassis 210, and one or more speakers 248 in the headrest 246.

In certain embodiments, the drive system 212 may include two articulated wheels (or wheel pairs) 214 and a caster wheel (or wheel pair) 216 (as depicted in FIG. 2A). Each articulated wheel 214 may be coupled to a drive motor. The control system 230 may drive the articulated wheel 214 at different speeds in order to steer, direct, navigate, etc. the patient transportation robot 200 to different procedure hubs 120 within the treatment room 110 (also known as differential steering). Additionally, each articulated wheel 214 may be independently steered by the control system 230. For example, the control system 230 may steer the patient transportation robot 200 according to a particular steering technique, such as Ackermann steering, active-front-and-rear steering, spinning, crab steering, etc. Other configurations of the drive system 212 are also supported, such as two (2) articulated side wheels 214 with front and rear casters to provide balancing support for the chassis 210, etc.

In some embodiments, the drive system 212 may include a motor that is coupled to a rail located on the floor of the ophthalmic injection facility 100. The rail forms a loop that passes through the diagnostic hub 130, the anesthetic hub 140, the patient interface hub 150, the antiseptic hub 160, the treatment hub 170, and the patient loading/unloading area 180. The control system 230 may be configured to translate the patient transportation robot 200 along the rail using the motor.

The proximity sensors 222 may detect objects that are close to the chassis 210 of the patient transportation robot 200 without making physical contact with those objects. The proximity sensors 222 generate proximity sensor data (signals, etc.) that are provided to the control system 230. The proximity sensor data may include measurements of the distance between the proximity sensor 222 and the object. The proximity sensors 222 may include ultrasonic sensors, infrared transceivers, photoresistors, LED sensors, light detection and ranging (LIDAR) sensors, etc. In certain embodiments, the patient transportation robot 200 may include a number of proximity sensors 222 that are distributed around the chassis 210 to provide up to 360° of detection coverage. For example, four proximity sensors 222 may each provide a 90° field-of-view, three proximity sensors 222 may each provide a 120° field-of-view, two proximity sensors 222 may each provide a 180° field-of-view, etc.

The line-following camera 220 is mounted to the front of the chassis 210 at a downward angle (such as 30°, 45°, 60°, etc.), and generates image data that are provided to the control system 230. The image data may be used for navigation as well as other purposes, such as object detection, etc. For example, the line-following camera 220 may detect the navigation line 112 on the floor of the ophthalmic injection facility 100 that delineates the path or track that link the procedure hubs 120 and the patient loading/unloading area 180. The navigation line 112 may be detectable in the visible spectrum, the infrared spectrum, or both. In certain embodiments, the line-following camera 220 may capture images at a modest resolution (such as 2 megapixel (MP), 4 MP, 6 MP, etc.) and a sharp focus. In some embodiments, the line-following camera 220 may be an infrared camera with an infrared light source (such as an LED).

FIG. 2B depicts a block diagram of the patient transportation robot 200 depicted in FIG. 2A, in accordance with embodiments of the present disclosure.

The control system 230 is coupled to the drive system 212, the line-following camera 220, the PI system vacuum pump 250, and the touchscreen display 260. In certain embodiments, the control system 230 may include a processor 232 coupled to a memory 234, a wireless transceiver such as a WiFi transceiver 236, and a GPS receiver 238. The WiFi transceiver 236 is coupled to the WiFi antenna 224, and the GPS receiver 238 is coupled to the GPS antenna 226. The WiFi antenna 224 and the GPS antenna 226 may be mounted to an external surface of the chassis 210 to improve signal transmission and reception. In some embodiments, the WiFi transceiver 236 and the GPS receiver 238 may incorporate antennae within their respective form factors.

Generally, sensor data may include image data generated by the line-following camera 220 and/or location data generated by the GPS receiver 238. In some embodiments, the control system 230 may be coupled to the proximity sensors 222, and the sensor data may include the image data from the line-following camera 220, the location data from the GPS receiver 238, and proximity data generated by the proximity sensors 222. In some embodiments, the control system 230 may be coupled to the speakers 248 to relay verbal instructions from, or reassurance to, the patient from the control computer 190 at the nurse’s station 105.

In certain embodiments, in response to receiving a travel command, the control system 230 may navigate from one procedure hub 120 to another procedure hub 120 based on sensor data, such as the image data from the line-following camera 220 and the location data from the GPS receiver 238. Similarly, in response to receiving a travel command, the control system 230 may navigate from the patient loading/unloading area 180 to the diagnostic hub 130 based on sensor data, and from the treatment hub 170 to the patient loading/unloading area 180 based on sensor data. As described above, the patient transportation robot 200 may navigate along the navigation line 112 on the floor of the treatment room 110.

A map of the treatment room 110 may be stored in the memory 234 of the control system 230, which may include the navigation line 112, the location and dimensions of the procedure hubs 120, and the walls of the treatment room 110, as well as any obstacles in the treatment room 110. The control system 230 (such as the processor 232) may process the sensor data and execute one or more models (such as machine learning models, etc.), etc., to determine a current location based on the sensor data and the map, determine a desired location based on the map, navigate to the desired location based on the sensor data and the map.

FIG. 2C depicts a UI 270 for the patient transportation robot 200 depicted in FIG. 2A, in accordance with embodiments of the present disclosure.

In certain embodiments, the UI 270 may be presented on the touchscreen display 260 of a patient transportation robot 200. The UI 270 may include a patient tab 272 that presents information related to the patient currently occupying the patient transportation robot 200. The information may include patient information, such as the patient’s name 273, etc., diagnostic measurements, such as IOP measurement 274, the patient’s treatment plan 275, patient notes 276, the ophthalmic surgeon’s name 277, etc.

In some embodiments, the UI 270 may be include controls and data 278 that are associated with the administration of the anesthesia to the patient prior to the ophthalmic injection, such as a start widget, an elapsed time display bar, a patient ready icon, etc.

In some embodiments, the UI 270 may be include control widgets 279 along the bottom of the patient tab 272 to access additional displays of information related to the patient, such as a detailed patient history, OCT data, biometric data, a detailed treatment plan, etc.

The information and data associated with the UI 270 may be stored in the memory 194 of the control computer 190, transmitted over a wireless link to the control system 230 of the patient transportation robot 200, and then presented by the processor 232 on the touchscreen display 260.

FIG. 3A depicts an ophthalmic injection robot 300 for the ophthalmic injection facility 100, in accordance with embodiments of the present disclosure.

In certain embodiments, the ophthalmic injection robot 300 includes a docking assembly 302 that receives a portion of the head 304 of a patient and covers one or both eyes 306 of the patient. The docking assembly 302 may include some or all of a forehead rest, cheek rests, temporal rests, chin rest, or rests for engaging other portions of the head 304 of the patient, some or all of which may be adjustable. The docking assembly 302 may include structures for clamping or otherwise retaining the head 304 of the patient, such as one or more clamping actuators 308 for pressing pads 310 against the patient’s head 304, a headband encircling the patient’s head 304 and fastened to the docking assembly 302, or other structures. Additionally, the PI device 500 may be coupled to the docking assembly 302 to prevent movement of the eye 306 to be treated.

The docking assembly 302 includes a robotic arm 312 for moving the docking assembly 302 into alignment with the head 304 of patients of various sizes. The robotic arm 312 may be understood with respect to X, Y, and Z direction, where the Z direction is substantially (e.g., within 2 degrees of) parallel to the direction of gravity and the X and Y directions are substantially (e.g., within 2 degrees of) perpendicular to the Z direction and to one another. The robotic arm 312 is configured to move the docking assembly 302 in the X, Y, and Z directions as well as one or more rotational degrees of freedom, such as rotation about an axis substantially (e.g., within 2 degrees of) parallel to the X, Y, and/or Z direction.

The robotic arm 312 may include a rotational joint 314 coupled to a base 316. The base 316 is coupled by elbow joint 318 to a link 320. Elbow joint 322 couples link 320 to link 324. Elbow joint 326 couples link 324 to link 328. Elbow joint 330 couples link 328 to link 332. Link 332 may be coupled by rotational joint 334 to the docking assembly 302. Each of the illustrated joints 314, 318, 322, 326, 330, 334 has a corresponding actuator for inducing movement of the joint. The robotic arm 312 may have at least five (5) degrees of freedom (DOF). For example, the illustrated robotic arm 312 has six (6) DOF. The robotic arm 312 may be embodied as a commercially available serial robotic arm. The robotic arm 312 may also be implemented as linear actuators, such as linear actuators implementing movements in the X, Y, and Z directions as well as one or more rotational actuators inducing rotation about one or more of the X, Y, and Z axes. For example, the robotic arm 312 may be embodied as gantry. Note that the illustrated size of the may be somewhat exaggerated relative to the size of the patient’s head 304 and may have a smaller relative size. For example, the robotic arm 312 may move the docking assembly 302 within a three-dimensional range of motion having dimensions in the X, Y, and Z directions that are less than 30 centimeters, 15 centimeters, or 10 centimeters.

The robotic arm 312 may be mounted to a support frame 336, such as by the rotational joint 314 mounting the illustrated robotic arm to the support frame 336. The support frame 336 may be mounted to a floor, wall, ceiling, movable cart, or other structure. The support frame 336 may include a docking interface 338 that is configured to couple the patient transportation robot 200 to the support frame 336 to prevent movement of the patient transportation robot 200.

The docking assembly 302 and actuators of the robotic arm 312 may be coupled to a control system 340. The control system 340 may be housed within the support frame 336 or elsewhere. The control system 340 may receive images from one or more cameras 342 in order to estimate a three-dimensional position of the patient’s head 304 and activate the robotic arm 312 to position the docking assembly 302 at or within a threshold distance of the patient’s head 304. The docking assembly 302 itself may include one or more cameras 344. Images from the one or more cameras 344 may be used by the control system 340 to perform fine adjustments to the position of the docking assembly 302. Alternatively, the docking assembly 302 may incorporate actuators that are controlled to perform fine adjustments of the docking assembly 302 based on one or more images from the one or more cameras 344.

The position of the docking assembly 302 itself may be determined by sensing a kinematic state of the robotic arm 312 using sensors incorporated into the joints 314, 318, 322, 326, 330, 334 or elsewhere in the robotic arm 312. Alternatively or additionally, the position of the docking assembly 302 may also be determined based on images from the one or more cameras 342, 344.

Although cameras 342, 344 are described as being used to estimate the position of the patient’s head 304 and possibly the docking assembly 302, other imaging or sensing modalities may be used such as light detection and ranging (LIDAR), radio detection and ranging (RADAR), ultrasonic sensing, or other type of sensor. The one or more cameras 344 may each be replaced with an OCT device, scanning laser ophthalmoscope, or other type of imaging device. An OCT device is particularly helpful for tracking the location of a needle during insertion, injection, and withdrawal.

FIG. 3B depicts a top view of a docking assembly 302 of the ophthalmic injection robot 300, while FIG. 3C depicts a partial side view of the docking assembly depicted in FIG. 3B, in accordance with embodiments of the present disclosure.

In certain embodiments, the docking assembly 302 may include a frame 400. The frame 400 may be embodied as a track, rail, or other structural member along which components may be fastened at various positions. The frame 400 may include one or more mounting structures 402 mounted thereto. Each mounting structure 402 has a staging assembly 404 mounted thereto. The staging assembly 404 includes one or more actuators that perform fine adjustments, relative to the precision of the robotic arm 312, of the position of the needle assembly 406 for inserting a needle 408 of the needle assembly 406 into the eye 306 of the patient in order to inject the ophthalmic drug.

In the illustrated embodiment, the staging assembly 404 includes an actuator 410 and an actuator 412 that are oriented substantially (e.g., within 2 degrees of) perpendicular to one another. The actuators 410, 412 may be linear actuators or the illustrated arcuate actuators 410, 412. For example, the actuators 410, 412 may define arcuate actuation paths that are each centered on a remote center of motion. For example, the remote center of motion may lie on the needle 408 or a path followed by the needle 408 when extended by an extension actuator 414, which is a linear actuator configured to extend and withdraw the needle 408 when performing injections. For example, the actuator 410 may be mounted to the mounting structure 402, the actuator 412 may be mounted to the actuator 410 and be actuated thereby along a first actuate path. The extension actuator 414 may be mounted to the actuator and may be actuated thereby along a second arcuate path that has the same remote center of motion as the first arcuate path, e.g., within 1 mm, 0.01 mm, or 1 micron. The needle assembly 406 may be mounted to the extension actuator 414 with the needle 408, or a line extending along the center of the lumen of the needle 408 lying on the remote center of motion, e.g., within 1 mm, 0.01 mm, or 1 micron.

The one or more clamping actuators 308 may be mounted to the frame 400. The clamping actuators 308 are configured to extend one or more pads 310 into engagement with the head 304 of the patient in order to reduce movement of the head 304 relative to the docking assembly 302. For example, there may be one pad 310 coupled to each clamping actuator 308 for comfortably clamping the head 304 of the patient.

Generally, the docking assembly 302 may be positioned relative to the head 304 of the patient using the robotic arm 312 and images from the one or more cameras 342. One or more images from the one or more cameras 344 of the docking assembly 302 may be used to determine the relative position of the eye 306 of the patient and perform fine adjustments using the robotic arm 312 based on the position. Once in position, the clamping actuators 308 may be activated to bring the pads 310 into engagement with the head 304 of the patient. Note that the position of the pads 310 may be asymmetric relative to the head 304 of the patient since the same docking assembly 302 may be used in two different positions to perform injections on the right and left eyes 306 of the patient. The actuation of the clamping actuators 308 may be guided by images from the one or more cameras 344. For example, the clamping actuators 308 may be used to adjust the relative positions of the docking assembly 302 and the patient’s head 304.

The docking assembly 302 may position the needle 408 on a line that intersects a point on the eye 306 of the patient at a prescribed position and angle, or within a tolerance of such a position and angle that is within the range of motion provided by the staging assembly 404. For example, when performing intravitreal injection, the prescribed position may be between 3 and 3.5 millimeters from the limbus for an aphakic eye and between 3.5 and 4 millimeters from the limbus for a phakic eye. The prescribed angle may be selected such that upon insertion of the needle, the needle avoids contact with the lens and retina while placing medication near the retina or area of the retina to be treated. The docking assembly 302 may include one or more electronic components in addition to the one or more cameras 344. The docking assembly 302 may include one or more fixation targets 420. Each fixation target 420 may be embodied as a static image, light source, screen for displaying a fixation target, or other device. A separate fixation target 420 may be provided for each eye 306 or a single fixation target 420 may be used for both right and left eyes 306. Alternatively, a single fixation target 420 may be mounted at different positions on the frame 400 for different eyes 306. In some embodiments a single fixation target 420 may be centrally located to be used for both eyes 306, so that each eye 306 may be directed toward the nose of the patient in order to expose the sclera for receiving an injection. Alternatively, a single screen implementing the fixation target 420 may display a fixation target at a different location for each eye 306. The location of the fixation target 420 may be adjusted using software executed by the control system 340 or by the ophthalmic surgeon in order to induce the patient to position the eye 306 at a desired angle.

The docking assembly 302 may include one or more intraocular pressure (IOP) sensors 422. The IOP sensor 422 may be a contact or non-contact sensor and may be used during intravitreal injection to ensure that the IOP of the patient’s eye 306 does not increase to unsafe levels. There may be separate IOP sensors 422 for each eye or a single IOP sensor 422 may be mounted at different positions on the frame 400 in order to measure the IOP of each eye 306. In certain embodiments, the ophthalmic injection robot 300 may transmit IOP data to the control computer 190, which then transmits the IOP data to the patient transportation robot 200 in the treatment hub 170 for presentation on the UI 270 of the touchscreen display 260.

FIG. 3D depicts a block diagram of the ophthalmic injection robot 300 depicted in FIG. 3A, in accordance with embodiments of the present disclosure.

The control system 350 is coupled to the robotic arm 312, the docking assembly 302, the cameras 342, 344, and the docking interface 338. In certain embodiments, the control system 350 may include a processor 352 coupled to a memory 354, a WiFi transceiver 356, and a network interface 358. The cameras 342, 344 generate image data, as described above.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A robotic system for an ophthalmic injection facility, the robotic system comprising:
one or more patient transportation robots, each patient transportation robot comprising:
a drive system,
one or more sensors configured to generate sensor data,
a patient interface (PI) system configured to be coupled to a PI device,
a chair configured to support a patient during an ophthalmic injection procedure, and
a control system configured to navigate to a treatment hub based on the sensor data; and
an ophthalmic injection robot located in the treatment hub, the ophthalmic injection robot comprising:
a support frame configured to receive each patient transportation robot,
a robotic arm coupled to the support frame, the robotic arm comprising one or more cameras and a docking assembly including a needle, and
a control system configured to:
position the docking assembly to couple the docking assembly to a PI device attached to an eye of the patient,
inject a drug through the needle into the eye of the patient, and
position the docking assembly to decouple the docking assembly from the PI device attached to the eye of the patient.

2. The robotic system of claim 1, wherein:
the PI device comprises an applanation lens, a suction ring, and a device connector;
the PI system of each patient transportation robot comprises a vacuum pump and a system connector configured to attach to the device connector; and
the control system of each patient transportation robot is further configured to provide suction to the suction ring using the vacuum pump when the device connector is attached to the system connector.

3. The robotic system of claim 1, wherein the support frame of the ophthalmic injection robot further comprises a docking interface configured to couple each patient transportation robot to the support frame.

4. The robotic system of claim 1, wherein the control system of each patient transportation robot is further configured to:
navigate to a diagnostic hub based on the sensor data;
navigate from the diagnostic hub to an anesthetic hub based on the sensor data;
navigate from the anesthetic hub to a patient interface hub based on the sensor data;
navigate from the patient interface hub to an antiseptic hub based on the sensor data; and
navigate from the antiseptic hub to the treatment hub based on the sensor data.

5. The robotic system of claim 4, wherein:
the drive system of each patient transportation robot comprises a plurality of articulated wheels; and
the one or more sensors of each patient transportation robot comprise a line-following camera, and a global positioning system (GPS) receiver.

6. The robotic system of claim 5, wherein the one or more sensors of each patient transportation robot further comprise one or more proximity sensors.

7. The robotic system of claim 4, further comprising:
a rail forming a loop that passes through the diagnostic hub, the anesthetic hub, the patient interface hub, the antiseptic hub, and the treatment hub;
wherein the drive system of each patient transportation robot comprises a motor coupled to the rail;
wherein the one or more sensors of each patient transportation robot comprise a global positioning system (GPS) receiver; and
wherein the control system of each patient transportation robot is further configured to translate the patient transportation robot along the rail using the motor.

8. The robotic system of claim 7, wherein the one or more sensors of each patient transportation robot further comprise one or more proximity sensors.

9. The robotic system of claim 4, further comprising:
a control computer comprising a processor, a memory, and a wireless transceiver configured to create a wireless communication link with each patient transportation robot,
wherein the processor of the control computer is configured to transmit commands to, and receive data from, each patient transportation robot over the wireless communication link, and
wherein the control system of each patient transportation robot comprises a processor, a memory, and a wireless transceiver.

10. The robotic system of claim 9, wherein the processor of the control computer is further configured to control movement of each patient transportation robot between the diagnostic hub, the anesthetic hub, the patient interface hub, the antiseptic hub, and the treatment hub.

11. The robotic system of claim 10, wherein the processor of the control computer is further configured to prevent more than one patient transportation robot to be present in the diagnostic hub, the anesthetic hub, the patient interface hub, the antiseptic hub, or the treatment hub at a same time.

12. The robotic system of claim 11, wherein:
the processor of the control computer is further configured to control a time of movement of each patient transportation robot between the anesthetic hub and the treatment hub; and
the time of movement is at least 10 minutes.

13. The robotic system of claim 12, wherein the one or more patient transportation robots includes at least 3 patient transportation robots.

14. The robotic system of claim 13, wherein the one or more patient transportation robots includes 5 patient transportation robots.

15. A patient transportation robot for an ophthalmic injection facility, the patient transportation robot comprising:
a drive system;
one or more sensors configured to generate sensor data;
a patient interface (PI) system configured to be coupled to a PI device;
a chair configured to support a patient during an ophthalmic injection procedure; and
a control system configured to navigate to a treatment hub based on the sensor data.

16. The patient transportation robot of claim 15, wherein:
the PI device comprises an applanation lens, a suction ring, and a device connector;
the PI system of each patient transportation robot comprises a vacuum pump and a system connector configured to attach to the device connector; and
the control system of each patient transportation robot is further configured to provide suction to the suction ring using the vacuum pump when the device connector is attached to the system connector.

17. The patient transportation robot of claim 16, wherein the patient transportation robot is configured to couple to a docking interface of an ophthalmic injection robot located in the treatment hub.

18. The patient transportation robot of claim 17, wherein the control system of the patient transportation robot is further configured to:
navigate to a diagnostic hub based on the sensor data;
navigate from the diagnostic hub to an anesthetic hub based on the sensor data;
navigate from the anesthetic hub to a patient interface hub based on the sensor data;
navigate from the patient interface hub to an antiseptic hub based on the sensor data; and
navigate from the antiseptic hub to the treatment hub based on the sensor data.

19. The patient transportation robot of claim 18, wherein:
the drive system comprises a plurality of articulated wheels; and
the one or more sensors comprise a line-following camera, and a global positioning system (GPS) receiver.

20. The patient transportation robot of claim 18, wherein:
the drive system comprises a motor coupled to a rail;
the one or more sensors comprise a global positioning system (GPS) receiver;
the control system of each patient transportation robot is further configured to translate the patient transportation robot along the rail using the motor; and
the rail forms a loop that passes through the diagnostic hub, the anesthetic hub, the patient interface hub, the antiseptic hub, and the treatment hub.
